(19) United States
(12) Patent Application Publication
(10) Pub. No.: US 2022/0347418 A1
(43) Pub. Date: Nov. 3, 2022

(54) PULSED OXYGEN SYSTEM AND PROCESS

(71) Applicant: Seabeck Holdings, LLC, Seabeck, WA (US)

(72) Inventors: James J Ruttler, Seabeck, WA (US); James L Ruttler, Rio Verde, AZ (US); Zuzana E Melherova, Seabeck, WA (US)

(73) Assignee: Seabeck Holdings, LLC, Seabeck, WA (US)

(21) Appl. No.: 17/725,956

(22) Filed: Apr. 21, 2022

Related U.S. Application Data
(60) Provisional application No. 63/177,571, filed on Apr. 21, 2021.
(60) Provisional application No. 63/197,262, filed on Jun. 4, 2021.
(60) Provisional application No. 63/222,976, filed on Jul. 17, 2021.

(51) Int. Cl. A61M 16/06; A61M 16/00

(52) U.S. Cl. CPC A61M 16/0677; A61M 16/024; A61M 2205/3306

(57) ABSTRACT

A system and process for an oxygen flow control system for supplemental oxygen is provided, including a system with an optical flow sensor and 3-way solenoid that operate to detect inhalation and deliver a microburst of oxygen that is electronically controlled based on one or more parameters.

### PRIORITY CLAIM

The present application is a non-provisional of and claims the benefit of and/or priority to the following applications under 35 USC 119 and/or 120: U.S. Provisional Patent Application 63/177,571 filed Apr. 21, 2021 (docket JIM-P-35); U.S. Provisional Patent Application 63/197,262 filed Jun. 4, 2021 (docket JIM-P-36); and U.S. Provisional Patent Application 63/222,976 filed Jul. 17, 2021 (docket JIM-P-37). All of the foregoing applications are incorporated by reference in their entirety as if fully set forth herein.

### BRIEF DESCRIPTION OF DRAWINGS

FIG. 1 is a system diagram of a smart pulse system, in accordance with an embodiment of the invention;

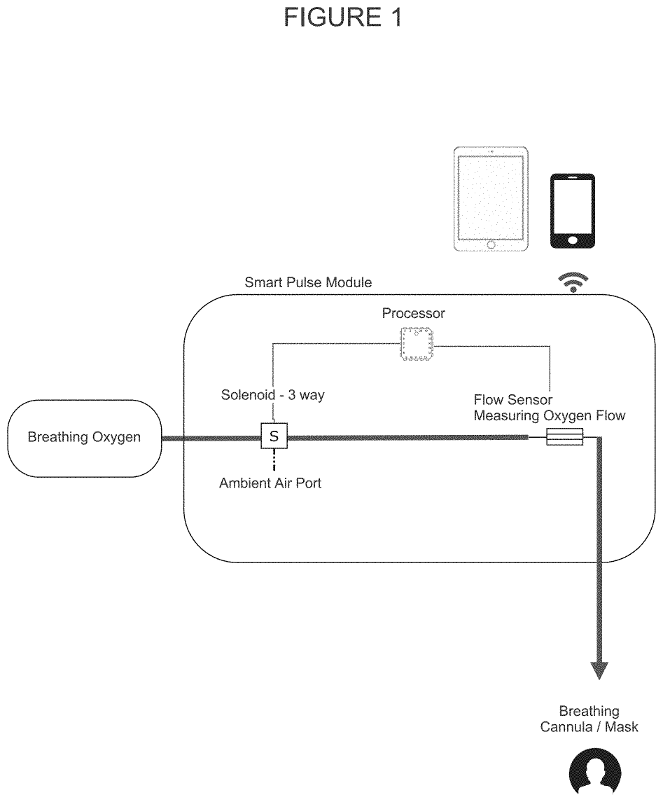

FIG. 2 is a system diagram of a smart pulse system, in accordance with an embodiment of the invention;

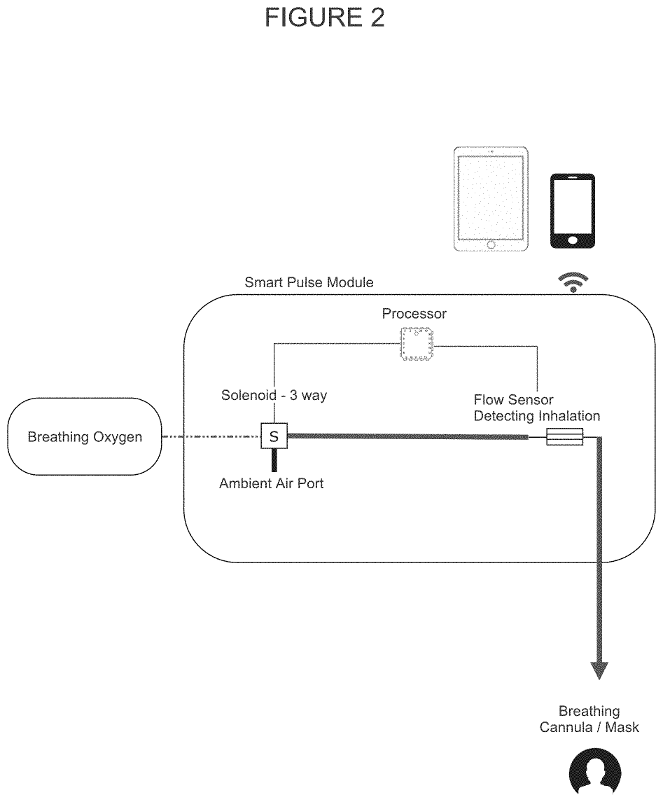

FIG. 3 is a system diagram of a smart pulse system, in accordance with an embodiment of the invention;

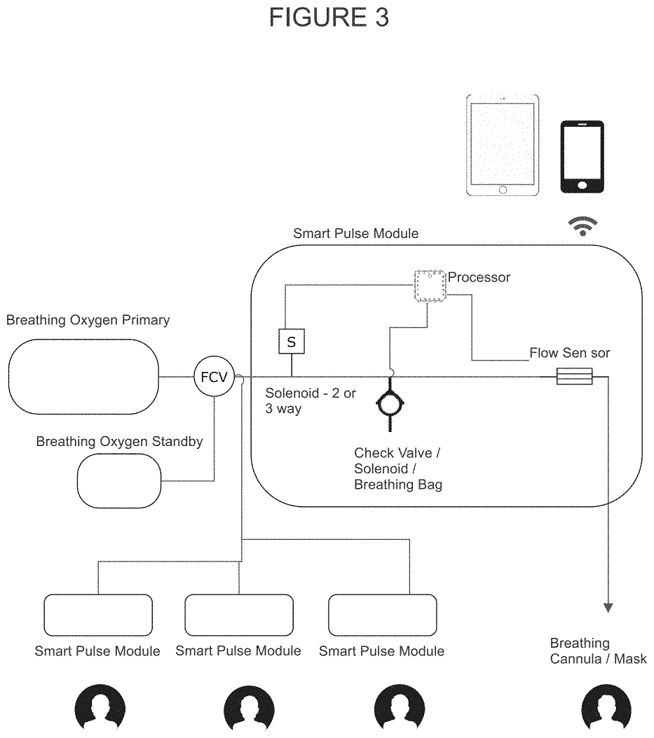

FIG. 4 is a system diagram of a multi-station smart pulse oxygen system, in accordance with an embodiment of the invention;

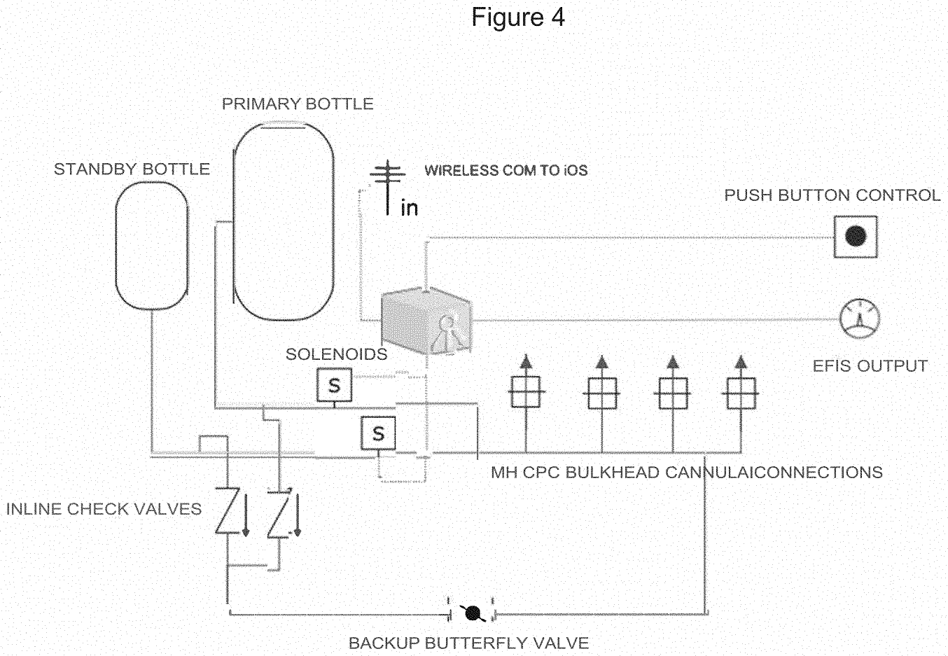

FIG. 5 is a flow diagram of a process for operating a smart pulse oxygen system, in accordance with an embodiment of the invention;

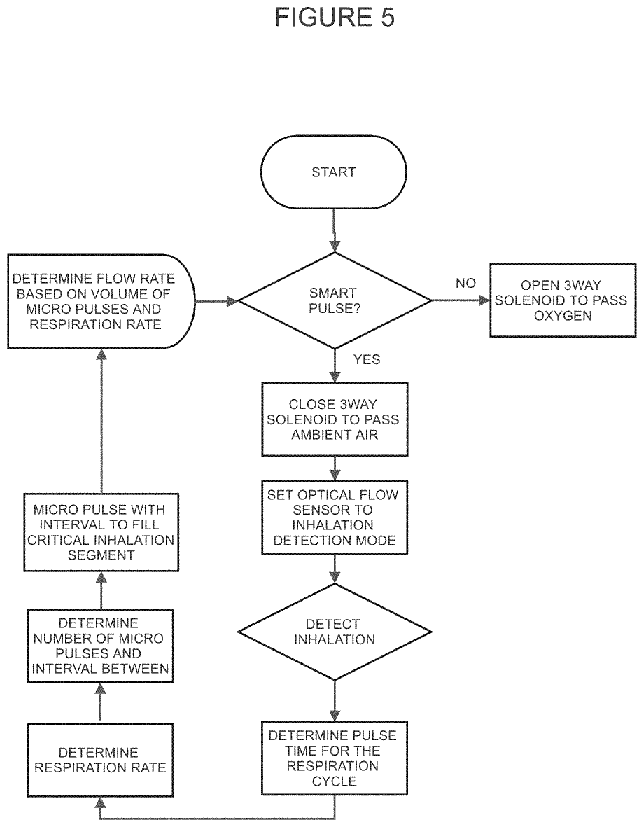

FIG. 6 is a flow diagram of a process for operating a smart pulse oxygen system, in accordance with an embodiment of the invention;

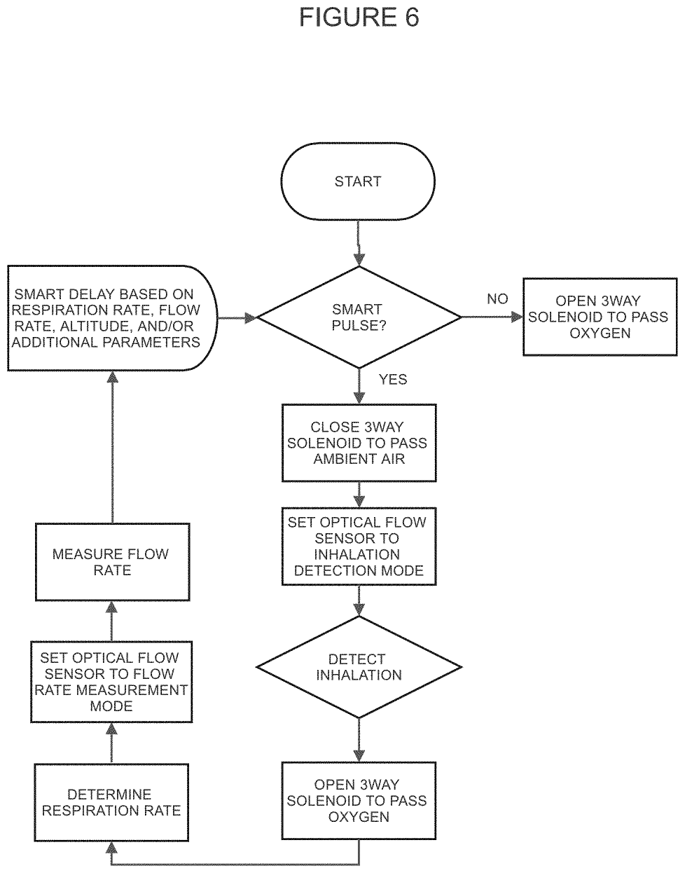

FIG. 7 is a flow diagram of a process for operating a smart pulse oxygen system, in accordance with an embodiment of the invention;

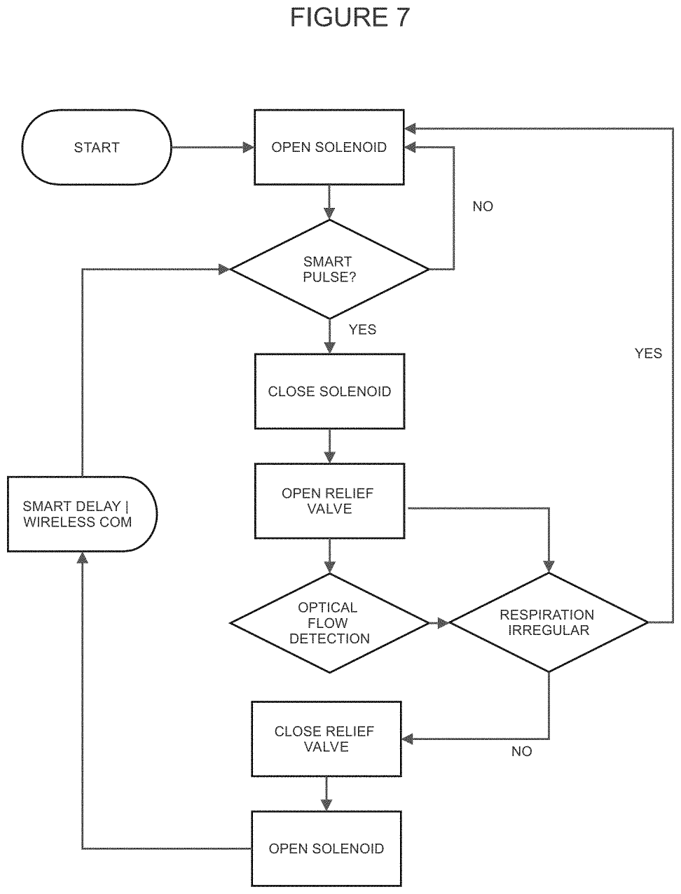

FIG. 8 is an illustration of dynamic microburst delivery of oxygen during a respiration cycle, in accordance with an embodiment of the invention;

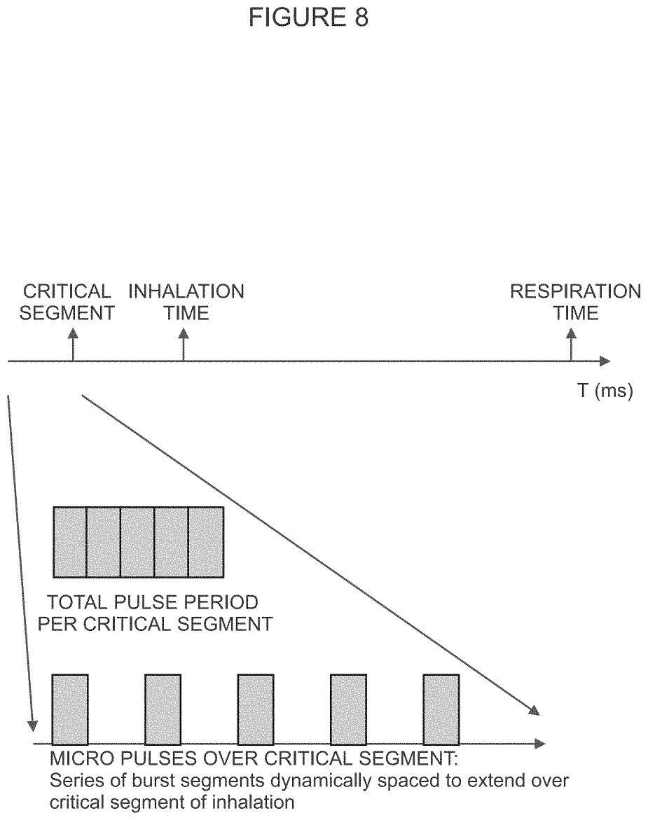

FIG. 9 is a flow diagram of a process for operating a dual bottle smart pulse oxygen system, in accordance with an embodiment of the invention; and

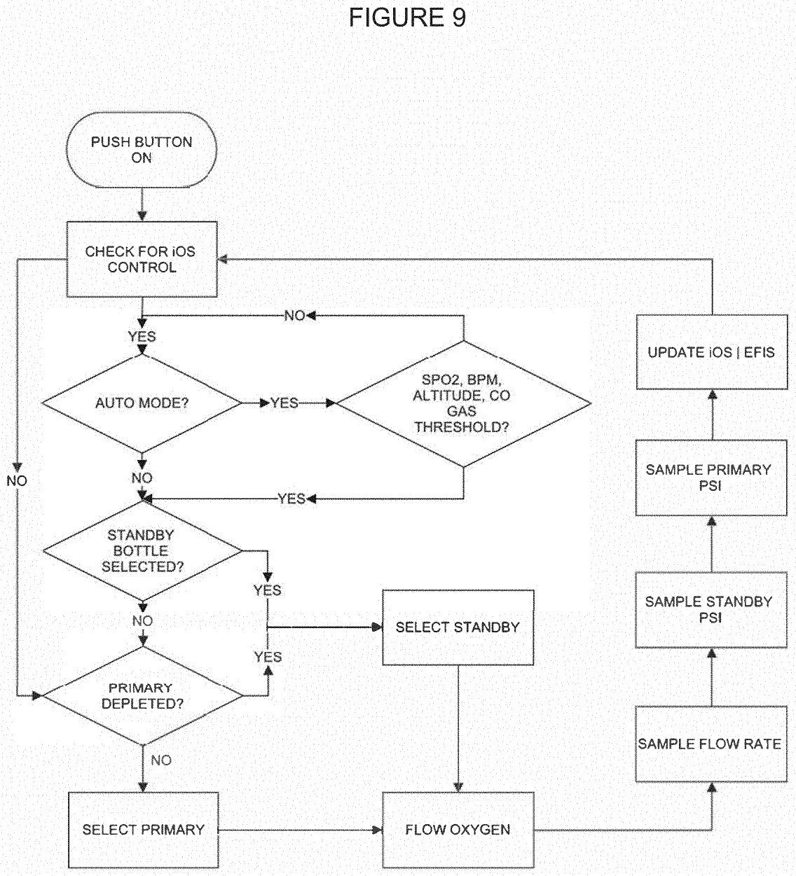

APPENDIX A are illustrations of various embodiments of smart pulsed oxygen systems.

### DESCRIPTION

A system and process for an oxygen flow control system for supplemental oxygen is described and illustrated herein.

A smart pulse/pulse demand type system is provided to conserve oxygen by delivering oxygen only during a portion of the inhalation period. When a user inhales, the oxygen is delivered over a period of time during the initial portion of the inhalation and then discontinued until the next inhalation. This method has been demonstrated to save substantial amounts of oxygen, such as extending the oxygen duration to over 8× that observed during non-conserving continuous flow modes.

Pulsed oxygen is known, but the system and process disclosed herein offers significant changes, improvements, and benefits as compared to the state of the art. For example, one leading pulsed oxygen system relies upon micro pressure changes to detect inhalation. When a user inhales through a nasal cannula, the pressure drops in the cannula line and that micro pressure drop can be sensed using a pressure sensor and processor. The processor then triggers the pulse of oxygen before repeating the process. This system has a number of disadvantages, including requiring a relatively strong inhalation to cause the pressure change and trigger the pulse. Also, the user cannula must be within close proximity (e.g. 5-8 feet) to the pressure sensor in order for the inhalation to be detected. There are additional deficiencies with this known leading pulsed oxygen system that stem from the absence of any flow rate information. Additionally, the pulsed oxygen of the art relies upon a single pulse per respiration interval, leading to a bolus of oxygen at one moment in time that cannot efficiently be absorbed.

We have conceived of an entirely different system and process to overcome the deficiencies in the art and significantly enhance the pulsed oxygen precision and reliability. The system includes a breathing oxygen supply that provides a source of oxygen to a 3-way solenoid valve. The 3-way solenoid valve passes the oxygen from the source to an inline electronic MEMS type flow meter before being delivered to the cannula and user for inhalation. When the 3-way solenoid is off, the oxygen delivery is stopped and does not pass through the 3-way solenoid or get delivered to the cannula and the user. When the 3-way solenoid is on, the oxygen delivery is started and the oxygen passes to the cannula and user for breathing. The 3-way solenoid has a second orifice that is open to ambient air. This second orifice is open with then oxygen is off and closed when the oxygen is on. That is, when the oxygen is off, the 3-way second orifice opens the cannula line to ambient air. During inhalation, the second orifice provides suction relief into the line where the oxygen normally travels enroute to the cannula and the user. Inhalation and exhalation is therefore freely open in the line, resulting in very small flow movements through the flow sensor. With the small movements of air via the flow sensor possible, the flow sensor coupled with a processor is able to detect the inception of an inhalation cycle and then turn on the oxygen supply to inject a pulse of oxygen into the respiration intake. What is further novel is the manner in which the oxygen is pulsed. The pulse is broken into a collection of individual micro pulses of approximately 20 milliseconds to inject a series of oxygen dosages into the inhalation stream. These micro bursts are better able to be absorbed into the bloodstream by the lungs because the oxygen is disbursed more evenly throughout the inhaled air.

Figures are included herein to illustrate this system arrangement. In summary, the breathing oxygen source provides oxygen to the cannula via the flow sensor when the 3-way solenoid is on, and the 2^{nd} orifice of the 3-way solenoid is blocked to prevent leakage of the oxygen except through the terminal end of the cannula. The flow sensor, being inline with the oxygen flow, can measure the oxygen flow rate (e.g. 0-2.5 LPM). Then, breathing oxygen can be discontinued by turning off the 3-way solenoid, resulting in the oxygen being stopped. At the same time, the 3-way solenoid second orifice is open to ambient air to permit the line to be instantly repurposed into an inhalation detection system. The second orifice provides the suction relief with the ambient air being able to pass into the line just downstream of where the oxygen was stopped, permitting prior oxygen and ambient air mixture to be moved through the flow sensor during inhalation. The amount of flow is very low during inhalation, but the flow sensor is operable to detect very low flow rates (e.g., 0-2.5 LPM). When the inhalation is detected through the flow sensor, the 3-way solenoid is activated resulting in the sealing off of the 2^{nd} orifice and opening of the breathing oxygen into the line for inhalation by the user via the cannula.

Each cycle of pulse is broken down into a series of micro pulses that span a portion or all of the inhalation cycle. In one embodiment, the amount of oxygen per pulse is calculated based on pressure altitude, physiological parameters such as SPO2 or heart rate, or environmental parameters such as carbon monoxide levels. Higher pressure altitudes, lower SPO2, higher heart rate, and higher carbon monoxide levels result in an increase in the volume of desired oxygen per inhalation cycle. Additionally, the respiration rate is calculated based on the time between a prior inhalation cycle, which can be averaged to smooth out changes. The respiration rate is then used to determine the respiration cycle and the respiration cycle is used to determine the inhalation segment time. For instance, with 12 breaths per minute, a 5 second respiration cycle is present and approximately ⅓ to ¼ of the 5 seconds is, on average, dedicated to inhalation.

Of the inhalation cycle, there is a known or understood portion that is critical to respiration and that is approximately ⅓ to ¼ of the inhalation period. So, for example, the inhalation segment of time may be determined to be 5/3/3=555 milliseconds. The desired dosage period is then spread out as micro bursts of, for example 20 millisecond bursts that span the entirety of the critical inhalation segment.

A substantially constant pressure and flow rate of oxygen is supplied to the input of the solenoid. Therefore, based on volume=flow rate*time, it is possible to select the volume of oxygen delivered per pulse period by varying the time that the solenoid is open as opposed to adjustment of the flow rate. As volume desired increases, such as by pressure altitude increases, the total time of oxygen desired to meet the volume also increases, with the rate being substantially constant. This total time per cycle of inhalation is spread out into micro bursts over the critical inhalation period. The intervals between bursts is dynamically changed to spread the constant micro bursts out over the period of time that is critical to inhalation. Note however that the micro burst time can also be adjusted, such as to provide a higher volume in a first micro burst that gradually decreases over the course of the series of micro bursts that constitute the total pulse.

So, for example, if a 100 millisecond volume of oxygen is desired over a determined 555 millisecond critical inhalation period, five 20 millisecond micro pulses can be spread out over the 555 milliseconds with approximately 90 milliseconds between the micro pulses. This example demonstrates how the micro pulses work for one set of parameters, but would change dynamically each breath. As the respiration rate decreased, the critical inhalation segment would increase. Likewise, as the physiological, altitude, or environmental parameters changed, so too would change the volume of pulsed oxygen and time of pulsed oxygen. This in turn would change the total number of micro pulses per inhalation. Furthermore, a user can tip the scales of the total volume as a percentage of standard per pulse or can modify the duration of each micro pulse, using voice control or user interface controls of an app. The computer processor then opens the solenoid using N or P channel transistor control, for instance, to execute on the micro pulse series and deliver the total volume of oxygen desired per pulse over a series of micro pulses for each dynamically determined critical respiration inhalation segment.

The micro pulses of each pulse duration may exceed the critical inhalation period. For example, 10 micro pulses of 20 milliseconds may be calculated for a 100 millisecond quick respiration cycle. This micro pulse request could not be satisfied, and the processor can revert to a single pulse over the 100 millisecond period.

The time duration of the micro pulse can be varied over the course of an inhalation segment, such as longer to shorter durations that are fixed or dynamically determined to spread out the entirety of the inhalation. The intervals between micro pulses can also be fixed or dynamically modified to ensure the micro pulses are spread out across a duration of the inhalation segment. The formula for determining the inhalation segment and critical portion of the inhalation segment can likewise be modified, with the fractions given being modified, such as per user or based on desired results. For instance, for a particular user, ½ of ½ of a respiration cycle may be selected to achieve best results of SPO2 over time, with micro pulses and volumetric oxygen amount being spread out over longer periods of time.

The processor resides electrically between the solenoid and the flow sensor and performs inter alia operations including switching the 3-way solenoid on/off and measuring the flow rate during different cycles of the solenoid state. The solenoid can be switched using a P-channel FET and the flow can be measured using an analog to digital converter or using I2C type communications. In continuous non-pulsed oxygen flow mode, the processor opens the 3-way solenoid to permit oxygen to flow from the oxygen source to the breathing mask. There is no pulsing required in continuous flow mode. In pulsed mode, the processor opens the 3-way solenoid to pass oxygen to the cannula via the flow sensor as described. The processor samples the flow rate from the flow sensor and determines the volume of oxygen flowing per unit of time (e.g. LPM) or can determine the flow rate mathematically based upon known input flow rate and pulse duration and respiration rate. This is the oxygen delivery rate that is important for metering the appropriate amount of oxygen to the user via the cannula. After a period of time (e.g, 500 ms or other as discussed herein), the processor turns off the 3-way solenoid to discontinue and conserve oxygen. The turning off of the 3-way solenoid opens the 2^{nd} orifice and exposes the downstream line to ambient air and suction relief. The processor then enters an inhalation detection mode to actively monitor the flow rates through the flow meter for a small increase that is indicative of an inhalation event. The threshold of flow that triggers the inhalation detection event is low and can be customized to ensure that the event coincides with actual inhalation. With the oxygen supply off in the line and the ambient air suction relief, the processor actively monitors the flow rate for inhalation. Once inhalation is detected, the processor then immediately triggers the 3-way solenoid to open for pulse or micro pulse operation. This results in the immediate passage of breathing oxygen via the 3-way solenoid, through the flow meter, and on to the cannula to fulfill the inhalation requirements of the user for oxygen. The 2^{nd} orifice of the 3-way valve is closed at the instant the 3-way solenoid is open for oxygen flow, by virtue of the operation of a 3-way solenoid. This prevents leakage of oxygen from the downstream system with the flow of oxygen commencing through the flow sensor and toward the terminal end of the breathing mask or cannula for consumption. This process can repeat indefinitely or under the control of the user.

The volume of oxygen required in the system invented herein is based upon two primary factors. The first is the pressure altitude of the system/user. In mountaineering, sports, aviation, skydiving or other activities of the atmosphere, the amount of oxygen available for inhalation decreases as the altitude increases. Pressure altitude is a more precise measurement of the available altitude because pressure altitude factors in weather changes to indicate the perceived altitude, which can fluctuate above and below a GPS actual altitude. The second is the respiration rate of the user. As the respiration rate increases the volume of oxygen per inhalation pulse decreases and conversely, as the respiration rate decreases, the volume of oxygen per inhalation pulse increases. The pressure altitude can be measured using a barometric pressure sensor. The respiration rate can be measured by the processor using the time between successive inhalation events. A common starting point for volume of oxygen required to support normal respiration is 1 LPM per 10,000 feet MSL pressure altitude, but this volume can be adjusted based also on user health and acclimatization.

Another factor that is possible to use in the system is feedback from actual pulse oximeter SPO2 blood oxygen readings. When the real-time oximetry readings show a decreasing rate of SPO2 over a period of time, the volume of oxygen can be increased until such point as the SPO2 is stabilized or increasing. The AITHRE ILLYRIAN wearable and continuously monitoring pulse oximeter provides the data points that are usable by the system herein to titrate oxygen delivery volume.

With the volume of oxygen determined the pulse timing can be adjusted in near-real-time, such as every pulse event to maintain the oxygen delivered volume as closely as possible to the desired oxygen volume. The timing often is in the range of milliseconds to seconds, which is easily tolerated by the processor, electronics, and the 3-way solenoid valve. For instance, a low respiration rate can result in an increase of pulse duration, as can an increase in altitude. As respiration rate intensifies or the altitude decreases, the pulse duration can become shortened. Additionally, the actual flow rate of oxygen through the system can be monitored to further adjust the timing of the pulse duration up or down.

The net result of this system arrangement and accompanying computer process is a titration of oxygen that is uniquely tailored to the environment and the user, with virtually no waste in oxygen other than that which is required to maintain healthy respiration. We are demonstrating very high levels of oxygen savings, in the range of greater than 8-10 times duration as compared to a continuous flow system. That is, with the same breathing oxygen supply, the system and process disclosed herein can extend the life of the oxygen supply by more than 8 times, with greater efficiency for users that are healthy or acclimatized and longer durations at lower altitudes. We have demonstrated low flow rates such as 0.36 CF/hour or 10 L/hour at 15 k-16 k feet MSL while maintaining SPO2% at or above 95%.

Furthermore, the system is able to detect inhalation much more sensitively than reliance on pressure drops in the line and there is no observable limitation on significant plumbing lengths due to the use of a MEMS or optical flow sensor. The inhalation can therefore be detected with light breathing and more quickly, enabling oxygen to be pulsed and delivered at the initial part of the inhalation period. This enables more oxygen to be delivered to the deepest parts of the lungs, with less waste and more efficiency, thereby maintaining high SPO2, high cognition, and other benefits of oxygen.

The system further can be used in a multi-place integrated system where the solenoid, processor, and flow sensor arrangement are duplicated for each desired user. The breathing oxygen source can be the same or duplicated and is fed to each of the lines that use distributed processing to independently monitor flow rates, detect inhalation, and adjust timing of the pulsed oxygen to fit the unique needs of the user/environment.

The disclosed system is further operable to communicate via BLUETOOTH or BLE or other wireless communication to a mobile device, smart phone, or tablet to provide a user interface to the system and/or to receive user inputs and sensor data. For example, the iOS devices, including iPhones and iPads, include barometric pressure sensors that can be used for altitude detection and can operate as a hub for collecting SPO2 and heart rate information from other devices, including the ILLYRIAN devices for each user. This data can be used to determine the pulse timing and inform and monitor operation of the system.

While the above system has been described and illustrated with a 3-way solenoid valve, it is also within the scope of this disclosure to use two separate 2-way solenoid valves. In the case of two 2-way solenoid valves, the first solenoid would control the flow of supplemental oxygen while the second solenoid valve would control the opening of the downstream plumbing 235 line to ambient air. We further disclose that the same effect of vacuum pressure release can be accomplished with an inline deflatable pouch/bag that is approximately the size of a fingertip. This bag can collapse upon inhalation to permit just enough movement of gas through the flow meter to detect the beginning of inhalation.

The cannula mask can be a nasal cannula with one or two prongs for oxygen delivery to the nostril(s). Alternatively, a mask covering can be used as well, as is required for certain aviation flights above 18 k MSL. It is also within scope to implement the pulse system and process disclosed herein within a helmet, boom cannula, mouthpiece, aviation headset, facial covering, or other device.

Two oxygen bottles are provided with the first being a primary and the second being standby. The primary and standby oxygen bottles have supply lines that feed to two solenoid valves or a single three-way solenoid valve. Each of the primary and standby bottles include an electronic pressure sensor. The solenoid output lines join together downstream to feed through an optical flow sensor. The output of the optical flow sensor is fed to one, two, three, four, or more quick connect ports for connecting to one or more cannula. A processor and wireless communication unit are electronically interconnected and wirelessly connected to one or more devices. An electronic push button is provided to turn on and off the system.

In operation the push button is activated to turn on the flow control system. The processor samples the pressure in the primary and standby bottles. When the primary bottle has sufficient oxygen, such as more than 50 PSI, the solenoid associated with the primary oxygen bottle is opened and the standby oxygen bottle and associated solenoid is closed. When the primary oxygen bottle pressure drops to an insufficient level, the standby solenoid associated with the standby bottle is opened automatically to permit oxygen flow. The flow is therefore uninterrupted when the primary bottle is depleted. The oxygen flow is verified through the optical flow sensor. Pressure for the standby and primary oxygen bottle, flow rate, and an indication of which bottle is active are provided wirelessly to a mobile device or tablet and/or via one or more serial or analog outputs to digital instrumentation, such as the Garmin G3x and Dynon Skyview type avionics systems.

For conservation and ease of management, the system can be automatically turned on and off to start and stop oxygen flow based on the following parameters: SPO2, heart rate, carbon monoxide level, and pressure altitude. These parameters can be defined in the instrumentation or the mobile device and when triggered result in the automated on and off control of the oxygen flow control system. This automation prevents oxygen flow except when needed based on the parameters being crossed, such as SPO2 under 92% or CO above 5 ppm or BPM heart rate above 100 bpm or pressure altitude of more than 5000 feet MSL at night or 8000 feet MSL day.

As safety and failure mode, in the event of uncertainty or lost communication with instrumentation, the oxygen can automatically default to the on and flowing state.

Individual pulse modules can be provided downstream of the flow control system. The pulse modules turn on and off with inhalation and exhalation, respectively, to conserve oxygen. An optical flow sensor detects inhalation and a relief permits the flow of air through the optical flow sensor. The relief can be a 3-way valve that is open to ambient air, a miniature breathing bag that is collapsible, a second solenoid that opens to ambient air, or a low pressure one-way check valve. Upon inhalation, the relief permits air to flow through the optical flow sensor. Very small rates of flow are detectable by this sensitive optical flow sensor. Immediately upon detecting inhalation, a solenoid that controls the passage of supplemental oxygen provided from the flow control system opens for a preset or adjustable period. Oxygen passes during this time through the plumbing and the optical flow sensor out to a cannula for breathing. The solenoid then closes and the optical flow sensor detects the next inhalation to repeat the process.

When the relief is a 3-way valve, the closing of the solenoid also opens the ambient air passage to permit inhalation detection—otherwise a vacuum is established and no flow is detected during inhalation. When the relief is a miniature breathing bag, the bag collapses to permit inhalation flow detection. When the relief is a second solenoid, the solenoid must be opened and closed in opposite the supplemental oxygen solenoid to permit the oxygen to not escape.

Wireless communication can be accomplished during the period that the supplemental oxygen flow is pulsed as no detection of inhalation is necessary. This small time period can be used during each inhalation breath to send and receive information without disrupting active monitoring of breathing through the optical flow sensor.

The pulse duration can be adjusted automatically based on SPO2, heart rate, carbon monoxide, or pressure altitude to increase or decrease the volume of oxygen that is passed through to the cannula for breathing during each inhalation. This information can be received during each inhalation and applied as needed to the very next breath to active and ongoing fine tuning of volumetric oxygen supply.

A plurality of pulse modules can be provided with a single flow control system to permit one or more other individuals to independently have pulse control and oxygen conservation, tuned to individual inhalation and hypoxia parameters such as SPO2, HR, CO, and pressure altitude. Each individual pulse modules can be adjusted and set using the digital instrumentation or the mobile device.

Failure and safety modes provide that the pulse module is operating in constant flow mode when respiration rate is too high or too low or when no inhalation is detected after a period of time. Additionally, the pulse can be enabled only when SPO2 is monitored continuously to prevent the pulse from being too low for a given set of hypoxia conditions, such as a high altitude or low blood oxygenation. Therefore, the pulse module can default and be elastic toward the continuous flow operation with pulse being an optional mode that is carefully monitored for safety.

The flow control valve and the pulse module can be combined into a single device that provides the same functionality in a portable battery operated unit. Alternatively, ship power such as 12V power can be provided to power the device.

In aircraft pressurized oxygen lines running through the cabin to outlet ports and to cannulas can be dangerous in the event of a fire. The oxygen in this case would significantly accelerate the first and decrease the odds of survival or fire extinguishment. The pulsed system described herein eliminates pressure in the plumbing lines between the pulse module and solenoids and the cannula, except in the case when inhalation and exhalation are detected. In the absence of an exhalation event (zero or below a threshold flow) and an inhalation event (above a threshold event), the solenoid remains closed, and no oxygen is loaded into the supply lines. The absence of oxygen removes the acceleration risk as no oxygen would flow if breathing was not present. Thus, in an event of a fire, a burned cannula or supply line would separate the breathing line and prevent further detection of inhalation. No oxygen would flow. The flow control module can be fire insulated with metal or fireproof material. Additionally, an auto shutoff fire protection valve can be placed on the output port of the regulator feeding the pulse module. The net effect of these properties means that lighter plastic tubes can be used more easily and safely than heavier metal plumbing lines that are prone to fail and leak.

Plumbing lines and cannula lines can fail or cannulas can be inserted but persons may bypass the same with mouth breathing. The result of any of these failures is the same—no supplemental oxygen inhalation. The present disclosure monitors the respiration rate and if none is detected when expected, alerts can be provided that no breathing is present. The alerts can be in the form of the button light turning off or flashing, audible tones of embedded buzzers, and/or Siri type alerts or popups via an associated app. For instance, in the case of a pilot flying an aircraft, the lack of breathing detected over a course of 30 seconds, for example, can result in a series of warnings to check the oxygen supply line or cannula.

1. A system comprising:
a processor;
a 3-way solenoid;
an optical flow sensor;
one or more plumbing lines configured to route oxygen from a source through the 3-way solenoid and the optical flow sensor.
